(19) United States
(12) Patent Application Publication
(10) Pub. No.: US 2016/0136442 A1
(43) Pub. Date: May 19, 2016

(54) MEDICAL DEVICE WITH PERSONALIZED THERAPY PROTOCOLS

(71) Applicant: TrioWave Technologies, Fremont, CA (US)

(72) Inventor: Huiyou Zhu, Fremont, CA (US)

(21) Appl. No.: 14/939,438

(22) Filed: Nov. 12, 2015

Related U.S. Application Data
(60) Provisional application No. 62/079,143, filed on Nov. 13, 2014.

(51) Int. Cl. A61N 1/372; A61N 5/06; A61N 1/36

(52) U.S. Cl. CPC A61N 1/37247; A61N 1/36014; A61N 5/06; A61N 2005/0626

(57) ABSTRACT

Devices and methods are described that generate personalized therapy protocols for home use of therapeutic medical devices. The devices include an intuitive user interface to guide user to select proper settings, a personalized therapy protocol-generating approach according to user selection and personal profile, and follow-up therapy protocol adjustments to achieve optimized results based on feedbacks collected from past treatments.

### RELATED PATENT APPLICATIONS

This application claims the benefit under 35 U.S.C. §119(e) of U.S. Provisional Application 62/079,143, filed Nov. 13, 2014, which is incorporated herein by reference in its entireties and for all purposes.

### BACKGROUND

Home based therapeutic devices, such as electrotherapy or phototherapy devices, usually come with preset therapy protocols, which require certain knowledge and experience to select and configure. Sometimes, mere knowledge and experience may not be adequate as optimal performance also depends on disease, condition, and physiological characteristics of the user, and some of such characteristics may require measurements that are beyond a home user's capability.

Electrotherapy is the practice of using electronic stimulation to help the body heal and recover after it has been damaged. With electrotherapy, a patient can control pain and speed up the healing process.

A common form of electrotherapy is transcutaneous electrical nerve stimulation (TENS). TENS uses small electrodes that can be fastened to the skin. The therapist administering the treatment can then adjust the amount of electricity that flows through the electrodes by a dial on the TENS device. This allows one to adjust the level of electricity to the comfort level and treatment level needed by the patient.

Electrotherapy works to heal in two main ways, reducing swelling and increasing circulation to the affected area. The reduced swelling allows more blood to rush to the areas that need healing. This also increases the circulation of the affected area. The increased blood flow allows oxygen and nutrients as well as healer cells of the body to start working on repairing the damaged tissue.

Electrotherapy works in pain management in two ways. Inside the body are large amounts of nerve cells and nerve pathways. These allow the brain to send signals to the body and the body to send signals to the brain. One of these signals is pain. Electrotherapy works by blocking the signal for pain, keeping the patient from feeling it. Electrotherapy also activates the body to produce endorphins. Endorphins are chemicals that act as the body's natural pain killers.

Phototherapy, also referred to as light therapy, consists of exposure to daylight or to specific wavelengths of light using polychromatic polarized light, lasers, light-emitting diodes, fluorescent lamps, dichroic lamps or very bright, full-spectrum light. The light is administered for a prescribed amount of time and, in some cases, at a specific time of day.

Phototherapy is commonly used for treating certain skin disorders such as mental disorder, acne vulgaris, eczema and neonatal jaundice and useful for control of arthritis-related pain and facial rejuvenation.

In addition, phototherapy which strikes the retina of the eyes is used to treat circadian rhythm disorders such as delayed sleep phase disorder and can also be used to treat seasonal affective disorder, with some support for its use also with non-seasonal psychiatric disorders.

Currently, therapeutic devices rely on users' own knowledge and experience in configuration (e.g., selection of therapy parameters) while being used. The devices themselves do not provide information or instructions to indicate whether a particular configuration is suitable.

### SUMMARY

This present disclosure describes devices and methods for generating personalized therapy protocols for home use of therapeutic medical devices, applying and configuring the protocols to carry out desired therapies, and adjusting and acquiring the protocols, such as through an internet source.

Therefore, the present technology provides, for home based personalized therapeutic devices, an intuitive user interface to guide user to select proper settings, a personalized therapy protocol-generating approach according to user selection and personal profile, and follow-up therapy protocol adjustments to achieve optimized results based on feedbacks collected from past treatments.

Furthermore, the present technology represents a new framework for therapeutic medical device performing personalized treatments. The medical device of this framework is able to implement all of the functions on a single hardware or software platform, or supplement functions (or components) on a local platform with function (or components) from remote platforms such as those on mobile or Internet resources.

In accordance with one embodiment of the present disclosure, provided is a therapeutic system which can be for home use, field use, or clinic use, comprising a signal generator for generating an electric or photo signal suitable for conducting a therapy, a graphic electronic display, one or more electrodes for applying the therapy and/or measuring a biological feedback, a processor and program code which, when executed by the processor, configures the system to (a) retrieve a therapy profile for a user by: receiving a user input comprising a body position of the user and a symptom or desired therapy at the position; querying a therapy protocol store with the user input, which therapy protocol store comprises a plurality of body positions and symptoms or desired therapies at each position, and, for each pair of body position and symptom or desired therapy at the position, one or more therapy protocols, wherein each therapy protocol comprises a therapy type, a therapy duration, and a therapy strength; and retrieving a therapy protocol from the therapy protocol store matching the symptom or desired therapy at the position in the input, (b) adjust a therapy protocol applied to a user by: receiving a biological feedback from the user undergoing an electric or photo therapy using the therapy protocol; determining whether the therapy protocol needs adjustment using a set of adjustment rules, which determination takes the biological feedback as input; and outputting an adjusted protocol based on the determination, and (c) provide graphic instruction to apply the therapy protocol retrieved from (a) and/or the adjusted therapy protocol determined from (b) by displaying an image and text indicating suitable placement of the therapeutic system according to the therapy protocol or adjusted therapy protocol.

In some aspects, the user input further comprises race, gender, age, height or weight of the user, and the therapy profiles in the metadata store further are associated with comprises race, gender, age, height or weight.

In some aspects, the adjustment determination further takes race, gender, age, height or weight of the user as input.

In some aspects, each therapy protocol further comprises a contraindication or warning.

In some aspects, the biological feedback comprises biological impedance, blood pressure, electromyographic (EMG) data or electrocardiogram (ECG) data.

In some aspects, the therapy is an electric therapy. In some aspects, each therapy protocol comprises a waveform, a duration, an amplitude, an intensity, a polarity and a frequency.

In some aspects, the therapy protocol store is located on a remote server.

In some aspects, (a) further comprises retrieving a historic therapy record of the user and querying the therapy protocol store with the record. In some aspects, the historic therapy record is located on a server or personal device that is insulated from the therapy protocol store.

### BRIEF DESCRIPTION OF THE DRAWINGS

Provided as embodiments of this disclosure are drawings which illustrate by exemplification only, and not limitation, wherein:

FIG. 1 illustrates a therapy protocol store that takes user input for identifying a suitable protocol for a user;

FIG. 2 illustrates a process in which a protocol is selected, personalized, and executed;

FIG. 3 shows a few different module configurations in terms of their relative locations;

FIG. 4 shows internal of Data Store of Control Unit for minimum data support in one embodiment; and

FIG. 5 is a flow chart of basic interactive therapy protocol selection and execution process.

It will be recognized that some or all of the figures are schematic representations for exemplification and, hence, that they do not necessarily depict the actual relative sizes or locations of the elements shown.

### DETAILED DESCRIPTION

The present disclosure, in one embodiment, describes systems and methods to generate therapy protocols to be used with home-based therapeutic medical devices to perform personalized and optimal treatments. The systems and methods can adjust therapy parameters and generate device placement instructions according to user input and/or treatment feedbacks collected during current or past treatments. Further, generation of the therapy protocol can take input from treatment information or protocols saved in a protocol store that is provided by a vendor or shared by other users.

Thus, in one embodiment, provided is a personal therapeutic system that includes a signal generator, a graphic interface, one or more electrodes for applying the signal to carry out the therapy or measuring a biological feedback from a user, a processor, memory, and program code. The signal generator can be used to generate an electric or photo signal suitable for conducting a therapy. The graphic interface can be used to take input from a user and/or display instructions. The program code includes instructions, which when carried out, help implement various functions of the present disclosure. In some aspects, the device can include other components, such as a battery unit, a charging unit, a wireless communication unit, without limitation.

As used herein, the term “therapeutic system,” “therapeutic device,” are “medical device” refers to an electronic device that generates a signal which, when applied to a human subject non-invasively, intends to bring about a therapeutic benefit. The signal can be electronic, magnetic, mechanical, light whether visible or invisible, or sound whether inside or outside human's hearing range. A device of this nature that uses an electronic waveform to stimulate a physiological reaction in a human subject is commonly referred to as an electrotherapeutic device. Likewise, a light- or photo-therapeutic device generates exposure to daylight or to specific wavelengths of light, using polychromatic polarized light, lasers, light-emitting diodes, fluorescent lamps, dichroic lamps or very bright, full-spectrum light.

### Therapy Protocol Store

In some embodiments, the device of the present disclosure generates or retrieves and optionally personalizes a therapy protocol for use with the device. The therapy protocol can be generated by the user, shared by another user, or retrieved from a therapy protocol store. In accordance with one embodiment of the disclosure, therefore, a therapy protocol store is provided which includes, without limitation, a metadata store, a protocol body and/or a placement instruction store.

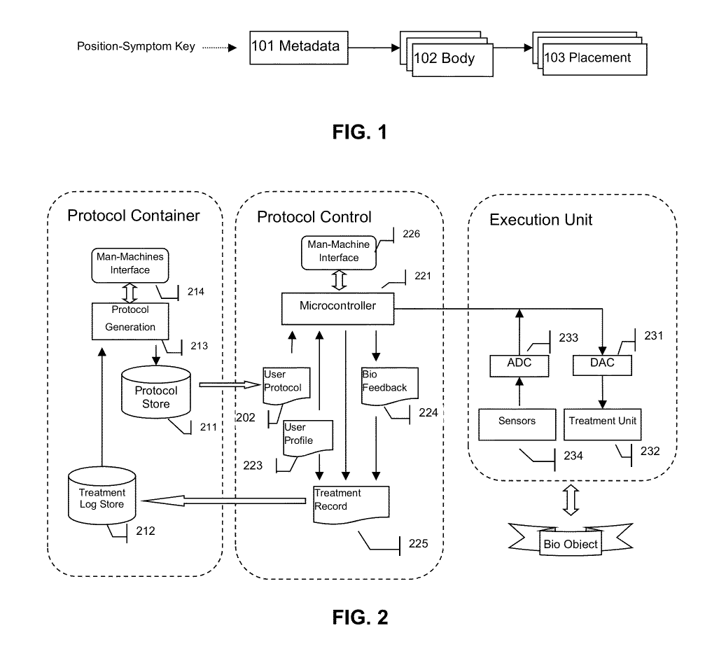

As illustrated in FIG. 1, to provide a user with an appropriate therapy protocol, the therapy protocol store can take an input from the user. In one aspect, the input includes a body position identified by the user and a symptom or desired therapy at the position. This combination of body position and symptom (or desired therapy) can serve as a key for data query, and is hereby referred to as “position-symptom key.”

The body position typically refers to an external body part, such as back, chest, hand, upper arm, eye, nose, face, or thigh, or a particular section of a body part. A symptom refers to a disease or condition that a user feels can be ameliorated or treated by the therapy. Non-limiting examples of disease include pain, itchiness, wound, allergy, arthritis, strain, sourness, numbness, tingling, dizziness, blurred vision, and infection.

A desired therapy, as used herein, refers to any therapy that can be provided by the medical device, whether or not it is applied to a disease or on a healthy subject. Non-limiting examples of desired therapy include electric stimuli to relax muscle, electric stimuli to stabilize nerve activity, sound signal to sooth brain activity, and photo treatment on skin.

The Metadata 101, for instance, can include body positions and symptoms or desired therapies at each position which in combination serve as a position-symptom key and can be used to search for a suitable therapy protocol. In addition, the Metadata 101 can further include targeted user profile (including race, sex, age, height and weight etc), indication for use, contraindication, warning, acquiring methods (free, try and buy, buy only), price, promotion (price and duration), and/or status (active, obsolete) for each therapy protocol.

The therapy protocol store can further include therapy protocols, which can be saved separately from the metadata store, such as in Body 102. Each therapy protocol, in some aspects, includes a therapy type, a therapy duration, and a therapy strength.

Therapy type of a therapy protocol can be, for instance, electrotherapy, phototherapy, radiotherapy or sound therapy, without limitation. The therapy duration can be the overall duration of the protocol, which can optionally include resting periods. The therapy strength can be measured or referred to in different units depending on the type of therapy. For instance, for electrotherapy, the strength relates to the amplitude of applied electric waveforms.

The protocol can include further parameters, such as intensity, duration, placement options, that can be tuned for therapy personalization. For instance, for electrotherapy, the protocol can further include polarity and/or frequency of the waveforms.

Suitable therapy protocols can be identified and retrieved by a user using an input that typically includes a position-symptom key, and optionally additional information provided by the user, such as gender, age, race, height, weight, allergy, disease, medication history, and/or whether the user is willing to pay for a protocol.

Once a suitable therapy protocol is identified by a user, the device can further provide graphic and/or text instructions to the user corresponding to the protocol. The instruction can take images and/or text from the Placement 103 unit of the therapy protocol store. The images in the Placement 103 unit can be pictures or illustration of body positions optionally with marking of proper positions of placement. The images, more generically, can also include video, audio, or animated images for improved instruction. In some aspects, the placement instructions are displayed on the graphic interface of the device.

Each entry in Metadata 101, in some aspects, can correspond to a plurality of entries in Body 102 to present alternative or combinational therapy for one particular position and symptom. For example, to one entry in Metadata 101 with position of low back and symptom of pain, the therapy protocol can have multiple Body 102 entries, one for electrotherapy, one for another method of electrotherapy, and another for phototherapy. Each of a multiple Placement 103 entries are then associated with each Body 102 entry to instruct how to operate the device for the corresponding Body 102 protocol.

As further described in more detail below, in addition to selection and personalization of the therapy protocols which typically occurs before a therapy begins, the present technology also enables adjustment of the therapy protocol during a therapy, in particular taking biofeedback into account.

The term “biofeedback” refers to information relating to physiological functions in a human subject, collected from the subject while or after the subject receives a therapy. Biofeedback can be gathered with methods known in the art, such as with contact (e.g., electrodes attached to a body part) or non-contact sensors. Non-limiting examples of such information include bio-impedance, blood pressure, electromyography (EMG), Electrocardiography (ECG), brainwaves, muscle tone, skin conductance, heart rate and pain perception.

### Protocol Selection and Execution Process

FIG. 2 illustrates a process designed to perform personalized therapy to achieve optimal result by adjusting treatment parameters and device placement according to past treatment biofeedback. As illustrated, the software program-implemented process includes a Protocol Container module, a Protocol Control module and an Execution Unit module.

The Protocol Container module generates and stores therapy protocols. Inside the module, there can be a Protocol Store 211 which can be a file, a folder or a database or the combination therefore, to store Metadata, Body and/or Placement of the therapy protocols. Treatment Log Store 212 contains past treatment records including user information, such as race, sex, age, height and weight, parameters and placement used in the past treatments, as well as biofeedback gathered from the past treatments.

Protocol Generation 213 can retrieve data from Treatment Log Store 212 for use by healthcare professionals. The retrieved data can be displayed on Man-Machine Interface 214 for the healthcare professionals to analyze past treatment settings and results, and generate new version of Protocol for either generic user or a particular user.

It is to be appreciated that, without limitation, the Protocol Container can reside in a local medical device or at a network resource such as a cloud computing platform.

The Protocol Control module includes a set of executable instructions, data and/or hardware components to control or manage protocol execution. As illustrated in FIG. 2, Microcontroller 221 is a central processing unit. User Protocol 222 is a cached copy of device supported protocols from Protocol Store 211. User Profile 223 stores user information including biological data such as age and race. Biofeedback 224 is a set of Body Policies of Protocols containing biofeedback gathered during or after treatments.

Treatment Record 225 is a placeholder to record treatment related data such as user profile, parameters applied, and biofeedback collected. Treatment Record 225 can be synchronized with Treatment Log Store 212 at a pre-specified interval or frequency.

The Execution Unit module accepts commands from Microcontroller 221 to convert digital signal to analog by DAC (digital-to-analog converter) 231, and directs Treatment Unit 232 to perform the therapy. For example, in an electrotherapy, Treatment Unit 302 can generate an electric waveform to be transmitted to electrodes affixed to a body part of a user. In a phototherapy, by contrast, Treatment Unit 232 can generate an array of selective LED emitting light energy to the user.

In some instances, during a treatment, in a pre-specified interval, Microcontroller 221 asks ADC (analog-to-digital converter) 303 to convert bio-signals gathered from a contact or non-contact Sensor 234 into digital for Bio-Feedback 224 generation.

### Protocol Adjustment

A therapy protocol selected for use by a user can be adjusted, before or during a therapy, to achieve optimal results. In general, the following three steps can be taken to implement the adjustment.

### A. Generation of a Basic Protocol

A basic protocol is one that is designed suitable for an average user to perform a generic therapy. A basic protocol takes the user's basic information (e.g., disease) into consideration during the design, but is not personalized to a particular user, that is, not taking the user's more specific information such as body weight, into consideration.

Protocol Generation 213 is a process that can be used by knowledgeable users or healthcare professionals to create therapy protocols and associated matching criteria and rules. Man-Machine Interface 214 is useful in this process serving as an interface between the users or professionals and the protocol store.

For example, a basic Protocol of position of low back and symptom of sprain, the creator should first define Metadata indicating version, position-symptom key, acquiring a method (e.g., downloading or purchasing a therapy protocol) and indication for use; and then, define a few Body indicating therapy like ElectroTherapy with required parameters and policy of parameter adjustment criteria against user profile (such as gender and race) and biofeedback from treatments, along with one or more Placement associated with the Body to illustrate how to place the Treatment Unit 232 properly to perform the Body.

### B. Protocol Personalization, Execution and Feedback Gathering

When a protocol is applied to a user, certain biofeedback can be collected from the user for further analysis and adjustments.

During execution, a basic protocol is copied from Protocol Store 211 and cached to User Protocol 222. After a user selects a position and a symptom on Man-Machine Interface 226, Microcontroller 221 loads a suitable protocol stored in Body 102 by matching the position-symptom key of Metadata from User Protocol 222. The microcontroller can also pulls user physiological data from User Profile 223.

Microprocessor 221, in some instances, can adjust the protocol parameters according to policies in Body 102 for the basic protocol using the physiological data from User Profile 222. The microprocessor can then send instructions of placement, pursuant to information stored in Placement 103, to the user to assist the user to configure or place the device, and send the protocol to DAC 231 and Treatment Unit 232 for execution. Periodically, Microprocessor 221 can measure BioFeedback 224 signals from ADC 233 and Sensors 234 to determine whether the therapy should continue.

Once the therapy is completed, Microprocessor 221 will save the protocol as applied to the user, which includes personalized parameters, along with the user's physiological data and collected biofeedback, to Treatment Record 225.

### C. Adjustment

Treatment Record 225, in some instances, is copied from Protocol Control Unit to Treatment Log Store 212 of the Protocol Container. Protocol Generation 213 can analyze and generate, either by a healthcare professional or by certain preset rules, a new version of protocol (adjusted protocol) according to the biofeedback data from Treatment Record 225. The adjusted protocol can then be saved to Protocol Store 211, optionally with a copy to User Protocol 222 for caching.

By repeating Steps B and C, an optimal protocol can be generated for a particular user.

### System Configurations

In the foregoing sections, the protocol generation, personalization, execution and adjustment process is described in general. It is to be appreciated that each module or portion of the process can be located differently relative to one another.

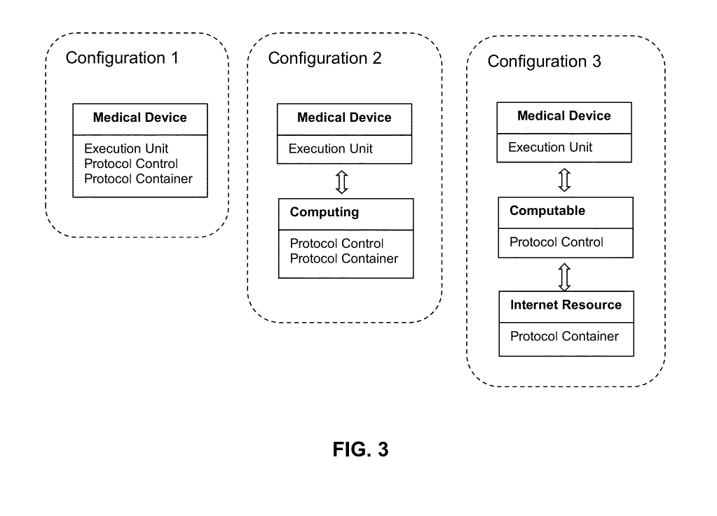

In some embodiments, the process can be partitioned into three units, a Protocol Container, a Protocol Control unit and an Execution Unit. Such partitioning increases flexibility in generating and adjusting personalized therapy protocols. FIG. 3 illustrates four different configurations of such partitioning.

Configuration 1 is a configuration in which the complete process is inside the medical device. Obviously, the treatment record base of Treatment Log Store for protocol parameter adjustment would be limited on this medical device wide treatment records. Besides, for a low-end medical device, its capability of processing power will restrain Protocol Container from advanced data analysis and complicated protocol adjustment/generation.

Configuration 2 can leverage Protocol Control and Protocol Container from high-end computing platform such as desktop computer or mobile device to perform advanced data analysis and complicated protocol generation. The high-end computing platform could maintain either wired or wireless communication link with Execution Unit on medical device. Moreover, the Treatment Log Store of Protocol Container could also be expanded to maintain more treatment records of Protocol Control gathered from multiple associated medical devices.

Configuration 3 further splits Protocol Container from computing platform to network based resourced such as cloud computing platform and big data technology, by expanding Configuration 1 and 2. By gathering treatment record from every network-enabled Protocol Control Unit, those records could be uploaded in either wired or wirelessly communication to Internet or Cloud based Protocol Container. Thus, with every treatment record uploaded from many and versatile medical device containing Protocol Control, the Treatment Log Store will gradually build up huge and versatile data for data mining, advanced analysis and more accurate parameter adjustment for personalized therapy protocol to specific position and symptom.

### Configuration of Device

The present disclosure further provides therapeutic medical devices suitable for generation and implementation of the disclosed protocols and processes of the disclosure. In one embodiment, the device can be used to achieve the following objectives.

A. Retrieving a protocol. The device includes a graphic or text user interface that enables a user to specify a body position and a symptom or a desired therapy at the position as a key for identifying and retrieving a suitable therapy protocol for the position and symptom.

B. Instructing device placement. Using the same or a different user interface, the device can instruct the user with respect to placing the device in a proper way for the therapy.

C. Protocol execution. With the retrieved protocol, the device can further adjust treatment parameters of the protocol according to information stored in the user profile.

D. Status update and protocol adjustment. The device can periodically gather status and biofeedback, with a remote or attached sensor, from the user's body. The gathered information can be used to monitor or control the therapy.

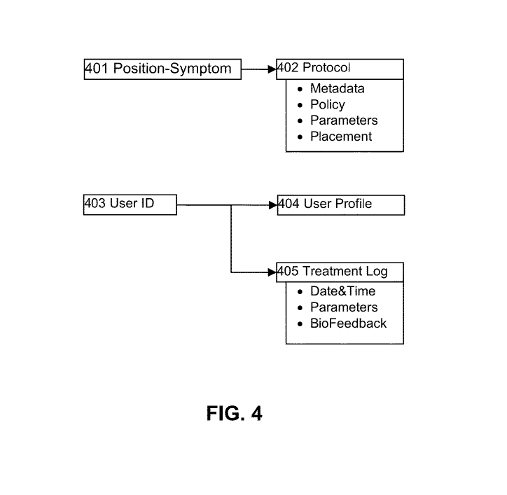

FIG. 4 illustrates the data structure of a data store organized to support therapy of protocol retrieval, parameter adjustment, and biofeedback gathering.

Protocol 402 is a list of therapy related data structures such as Metadata, Policy, parameters and pictures. Metadata contains applied position and symptom, version, indication for use, warning, contraindication, acquiring option (e.g., buy, trial to buy, or free download), price, and active status etc. for viewing, comparison and acquiring. Policy data contains some rules in adjusting therapy parameters such as 10% less of duration for child under 14. Parameter data contains duration, intensity, frequency or other technical data to perform this therapy. And Placement data contains some graphic or text to instruct how to place medical device properly before performing the therapy.

The protocol defined in Protocol 402 presents a generic/basic treatment protocol pattern to the symptom on specific position. As explained above, certain adjustments of parameters can be carried out later for a personalized treatment.

User Profile 404 is a pre-stored user profile containing personalized bio-data like sex, age, race and weight.

Treatment Log 405 stores every performed treatment including timestamp like date and time, adjusted parameters like intensity, frequency or duration according to user profile data, and bio feedback like water, protein, electrical conductance, pulse, or blood circulation during treatment.

User ID 403 is a key index to access specific user records of User Profile 504 and Treatment Log. Each user will be assigned a unique ID to access personalized data.

For an optimal personalized treatment, the parameters of protocol requires some adjustments by Policy of the protocol from Protocol 402 with User Profile 404 and Biofeedback of Treatment Log 405. Those adjusted parameters will be used for this personalized treatment and recorded into Treatment Log 405.

With special values, such as ‘Electro-All, of Position-Symptom 401 as key index, the Protocol 402 also allows user to access some protocol for advanced user to set or customize treatment parameters directly in own flavor without involving User Profile 504 for policy based adjustment.

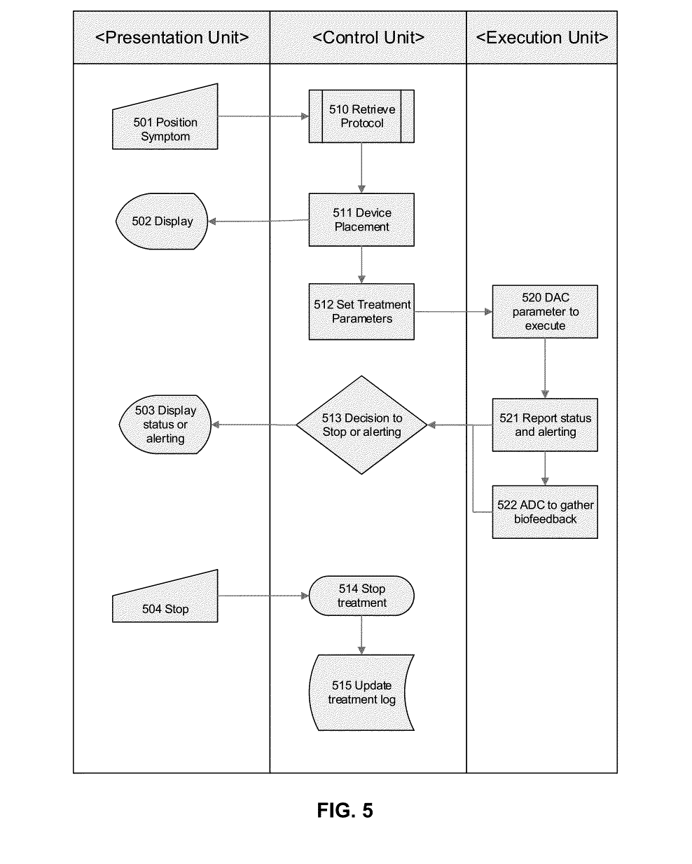

FIG. 5 illustrates a data and work flow, or process, for a personalized treatment in four stages.

Stage 1: Retrieving protocol 510—According to user selected Position and Symptom 301 from Presentation Unit, a key index of Position-Symptom 401, or in Symptom-Position order, will be used to retrieve suitable protocol from Protocol 402 container of Data Store.

Certain pre-qualification will be performed based on protocol Metadata to determine if the protocol could be adapted such as to pacemaker user. If not, warning message will be sent back Presentation Unit for consideration.

Stage 2: Device placement 511—The placement pictures or text instruction of medical device will be sent back to Presentation Unit to illustrate how to place medical device for next therapy.

Stage 3: In treatment—this stage consists of at least two processes;
Set Treatment Parameters 512 adjusts parameters according User Profile 404 to reflect a personalized setting of protocol;
DAC 520 will transform adjusted parameters from digital into analog signal and sent to Therapy Module to execute.

Step 4: Status update—With certain interval predefined, Micro Controller of Control Unit will query Execution Unit for status, such as low battery or over temperature, and bio feedback, such as pulse or conductance etc., gathered by ADC 522 for a Decision of Stopping, Continue, or Alerting 513.

The status or alerting will also be filtered and sent to Presentation Unit to Display 303. User can intervene treatment process by sending request of Stop 504 according to presented status or alerting to Stop treatment 514.

Whenever the treatment stops, the information of parameter used, duration, and biofeedback can be updated in treatment log 515.

### Intuitive User Interface and Instruction

The present technology provides an intelligent platform to provide a simplified and intuitive process for a user to receive personalized therapy. The intuitive user interface enables selection and optionally personalization of a therapy protocol with minimum user input. In some instances, the user only needs to specific a body position and a symptom or a desire therapy at the position. By contrast, in conventional technologies, a user is required to select technical parameters, which selection depends on a user's knowledge and experience of the treatment.

Thus, in one embodiment, on a graphic or text user interface of a device of the present disclosure, a user specifies a body position and a symptom (or a desire therapy). As described above, the device retrieves from a therapy protocol store a suitable therapy protocol, using the body position and system as input.

While applying the therapy protocol to a user, the device can further present, on a graphic user interface, picture or text to guide user to place the device (e.g., electrodes or photo emitters) at proper position for the therapy. The user can readily follow the instruction to apply the therapy.

During the therapy, in some embodiments, the device can adjust therapy parameters according to biofeedback collected from the user. This is particularly useful as the user's condition may alter during the therapy, or when the user has improperly entered body position or symptom. In some instances, the device can shut off when a therapeutic goal has been achieved or a therapy poses risk to the user.

The device can also adjust therapy parameters, at any time during a therapy, according to information in a user profile. The user profile stores basic user information, such as race, sex, age, height and weight, and historic treatment record, including biofeedback collected from the user.

Such an intuitive platform is useful for various types of therapeutic devices. Applicable therapy types include, without limitation, electrotherapy, phototherapy, radiotherapy or sound therapy. A therapy duration, in a therapy protocol, can be the overall duration of the protocol, which can optionally include resting periods. A therapy strength can be measured or referred to in different units depending on the type of therapy. For instance, for electrotherapy, the strength relates to the amplitude of applied electric waveforms. The therapy protocol selected can include further parameters, such as intensity, duration, placement options, that can be tuned for therapy personalization. For instance, for electrotherapy, the protocol can further include polarity and/or frequency of the waveforms.

### Therapy Methods

Methods of therapy are also provided. For instance, the device outputs waveforms according to a personalized therapy protocol that can be generated from the systems are applied to a human subject. Various parameters of the waveforms can be adjusted to suit the user or the particular disease or condition that the user has, such as back pain, arthritis at the knee, or wound on the skin.

A micro current electrotherapy entails sending relatively weak electrical signals into the body of an individual in need of the therapy. Such therapies apply small (e.g., between 1 and 50 microampere) electrical currents to nerves using electrodes placed on the skin. Micro current electrotherapies can be used in treatments for pain, age-related macular degeneration, wound healing, and tendon repair, without limitation. Many micro current treatments concentrate on pain and/or speeding healing and recovery. Micro current treatments are commonly used by professional and performance athletes with acute pain and/or muscle tenderness as they are drug-free and non-invasive, thus avoiding testing and recovery issues. They can also be used as a cosmetic treatment.

Phototherapies, radiotherapies, and sound therapies can be likewise configured and executed, to achieve therapeutic or cosmetic results.

Although the discussions above may refer to a specific order and composition of method steps, it is understood that the order of these steps may differ from what is described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure. Such variations will depend on the software and hardware systems chosen and on designer choice. It is understood that all such variations are within the scope of the disclosure. Likewise, software and web implementations of the present disclosure could be accomplished with standard programming techniques with rule based logic and other logic to accomplish the various database searching steps, correlation steps, comparison steps and decision steps.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed here. For example, the terms “comprising”, “including,” containing,” etc. shall be read expansively and without limitation. Additionally, the terms and expressions employed here have been used as terms of description and not of limitation; hence, the use of such terms and expressions does not evidence and intention to exclude any equivalents of the features shown and described or of portions thereof. Rather, it is recognized that various modifications are possible within the scope of the disclosure claimed.

By the same token, while the present disclosure has been specifically disclosed by preferred embodiments and optional features, the knowledgeable reader will apprehend modification, improvement and variation of the subject matter embodied here. These modifications, improvements and variations are considered within the scope of the disclosure.

The disclosure has been described broadly and generically here. Each of the narrower species and subgeneric groupings falling within the generic disclosure also form part of the disclosure. This includes the generic description of the disclosure with a proviso or negative limitation removing any subject matter from the genus, regardless of whether or not the excised material is described specifically.

Where features or aspects of the disclosure are described by reference to a Markush group, the disclosure also is described thereby in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Although the disclosure has been described in conjunction with the above-mentioned embodiments, the foregoing description and examples are intended to illustrate and not limit the scope of the disclosure. Other aspects, advantages and modifications within the scope of the disclosure will be apparent to those skilled in the art to which the disclosure pertains.

1. A therapeutic system, comprising a signal generator for generating an electric or photo signal suitable for conducting a therapy, a graphic electronic display, one or more electrodes for applying the therapy and/or measuring a biological feedback, a processor and program code which, when executed by the processor, configures the system to:
(a) retrieve a therapy profile for a user by:
receiving a user input comprising a body position of the user and a symptom or desired therapy at the position;
querying a therapy protocol store with the user input, which therapy protocol store comprises a plurality of body positions and symptoms or desired therapies at each position, and, for each pair of body position and symptom or desired therapy at the position, one or more therapy protocols, wherein each therapy protocol comprises a therapy type, a therapy duration, and a therapy strength; and
retrieving a therapy protocol from the therapy protocol store matching the symptom or desired therapy at the position in the input,
(b) adjust a therapy protocol applied to a user by:
receiving a biological feedback from the user undergoing an electric or photo therapy using the therapy protocol;
determining whether the therapy protocol needs adjustment using a set of adjustment rules, which determination takes the biological feedback as input; and
outputting an adjusted protocol based on the determination, and
(c) provide graphic instruction to apply the therapy protocol retrieved from (a) and/or the adjusted therapy protocol determined from (b) by displaying an image and text indicating suitable placement of the therapeutic system according to the therapy protocol or adjusted therapy protocol.

2. The system of claim 1, wherein the user input further comprises race, gender, age, height or weight of the user, and the therapy profiles in the metadata store further are associated with comprises race, gender, age, height or weight.

3. The system of claim 2, wherein the adjustment determination further takes race, gender, age, height or weight of the user as input.

4. The system of claim 2, wherein each therapy protocol further comprises a contraindication or warning.

5. The system of claim 1, wherein the biological feedback comprises biological impedance, blood pressure, electromyographic (EMG) data or electrocardiogram (ECG) data.

6. The system of claim 1, wherein the therapy is an electric therapy.

7. The system of claim 6, wherein each therapy protocol comprises a waveform, a duration, an amplitude, an intensity, a polarity and a frequency.

8. The system of claim 1, wherein the therapy protocol store is located on a remote server.

9. The system of claim 8, wherein (a) further comprising retrieving a historic therapy record of the user and querying the therapy protocol store with the record.

10. The system of claim 9, wherein the historic therapy record is located on a server or personal device that is insulated from the therapy protocol store.
